(19) United States
(12) Patent Application Publication
(10) Pub. No.: US 2017/0172738 A1
(43) Pub. Date: Jun. 22, 2017

(54) Cardiac prosthesis stabilization system

(71) Applicant: Safwan Kassas, Bloomfield Hills, MI (US)

(72) Inventor: Safwan Kassas, Bloomfield Hills, MI (US)

(21) Appl. No.: 15/530,270

(22) Filed: Dec. 16, 2016

Related U.S. Application Data
(60) Provisional application No. 62/268,373, filed on Dec. 16, 2015.

(51) Int. Cl. A61F 2/24

(52) U.S. Cl. CPC A61F 2/2409; A61F 2/2418; A61F 2210/0014; A61F 2220/0016; A61F 2230/0034

(57) ABSTRACT

According to the present teachings, a support structure for cardiac prosthesis is disclosed. The support structure can include an annular outer ring that can be used to stabilize/anchor devices used for valvular repair or replacement or left atrial appendage closure. Additionally, a valve repair device that can be used to treat cardiac valvular regurgitation. Further, a left atrial appendage closure device that can be used for left atrial appendage closure.

### CROSS-REFERENCE TO RELATED APPLICATIONS

This application claims the benefit of U.S. Provisional Application No. 62/268373, filed on Dec. 16, 2015. The entire disclosure of the above application is incorporated herein by reference.

### FIELD

The present disclosure relates to a prosthetic for the circulatory system, and more particularly to a Cardiac Prosthesis Stabilization System that can be used with a prosthetic mitral valve and left atrial appendage closure.

### BACKGROUND

This section provides background information related to the present disclosure which is not necessarily prior art.

Due to aging and disease, cardiac tissue such as a mitral valve and left atrial appendage can be damaged so it affects the health of a patient. Invasive action can be taken to address the damage because the damage can cause the mitral valve to leaking (regurgitation) or does not fully open (stenosis). Additionally, invasive action can be taken when there is prolapse that cannot be treated with medicine, or there is an infection (endocarditis). Because of the heart geometry as well as the loads associated with the mitral valve and surrounding cardiac tissue, mitral valve and left atrial appendage repair is traditionally preferable to replacement for patients with a leaking valve and many with narrowed valves. Repair traditionally has been less effective than desired, and as such, there is a need for improved treatments.

### SUMMARY

This section provides a general summary of the disclosure, and is not a comprehensive disclosure of its full scope or all of its features.

According to the present teachings, a prosthetic mitral valve is provided. The valve comprises a support device having an interior bearing ring and an exterior bearing ring configured to be selectively engaged with heart muscle adjacent to the mitral valve. The inner bearing ring defined a through hole having and internal cylindrical bearing surface. Coupled to the internal cylindrical bearing surface is a valve element that is configured to restrict blood flow in a direction. The interior and exterior bearing ring are coupled together using an elastic member.

According to another teaching, the first exterior ring comprises a metallic ring formed of nitinol or titanium. The ring has a muscle bearing surface having at least one of micro spikes, a calcium like roughened surface, a powder metal coated surface, configured to adhesively and sealably adhere to cardiac tissue.

According to another teaching, the first exterior ring comprises a fabric disc supported by a plurality of internal deformable members.

According to an alternate teaching, the first exterior ring comprises a knit fabric tube formed about the metallic ring.

According to an alternate teaching, the first exterior ring comprises an inflatable PTFE tube.

According to an alternate teaching, an exterior surface of the PTFE tube has protrusions formed of a “calcium like material” and/or nitinol.

According to an alternate teaching, the valve element comprises a nitinol stent.

According to an alternate teaching, the valve element comprises a plurality of deformable valve leaves configured to augment the blood restriction of the natural mitral valve.

According to an alternate teaching, the valve element comprises a plurality of deformable valve leaves configured to replace the blood restriction of the natural mitral valve.

It is envisioned that various valve material and designs can be used in conjunction with the coupling mechanism. Specific embodiments showed valve systems which entirely replaced existing valves. Other embodiments show mitral valve constructions which augment the functioning of the natural mitral valve.

According to the present teachings, the method for inserting a mitral valve according to the present teachings includes: 1—Mimicking calcified aortic stenosis pathology (manufactured calcium like deposits with similar physical characteristics to calcium will provide adequate friction and capture between the ring and the native annulus tissue and between the ring and the stent frame); 2—determining a size of the oversized anchoring nitinol ring; 3—providing a PTFE ring (tube) surrounding the Nitinol wire which can be filled with polymer that will undergo thermal change to a solid form upon exposure to body temperature; Optionally, Micro spikes (can under pressure embed in native tissue; and 5—Over sized stent in relation to ring.

1—Liquid polymer and before undergoing thermal hardening will push PTFE membrane outward to seal all peripheral device empty spaces and preventing PVL; 2—After undergoing thermal transformation the presence of pressurized hardened polymer materials will add more anchoring force between the ring and native annulus; and 3—the presence of a layer of polymer between the dynamic annulus and the stent frame will protect the stent frame from the direct deforming force of the dynamic annular morphological changes.

According to the present teaching, a “calcium like material” combined with outer ring and a nitinol cage to fixably couple a valve into the circulatory system.

According to the present teaching, these constructions can be used with a mitral valve repair or to close off the LAA ostium.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 represents a cross section of the heart having a mitral valve prosthetic according to the present teachings;

FIGS. 2a-2d represent cross sections of a portion of the implant shown in FIG. 1;

FIGS. 3a-3b represent top views of the implant shown in FIG. 1;

FIGS. 3c-3d represent cross sectional and perspective views of the implant shown in FIG. 1;

FIGS. 4a-4b represent side sectional views of the implant shown in FIG. 1; and

FIGS. 5a-5b represent views of an LAA implant according to the present teachings.

Corresponding reference numerals indicate corresponding parts throughout the several views of the drawings.

### DETAILED DESCRIPTION

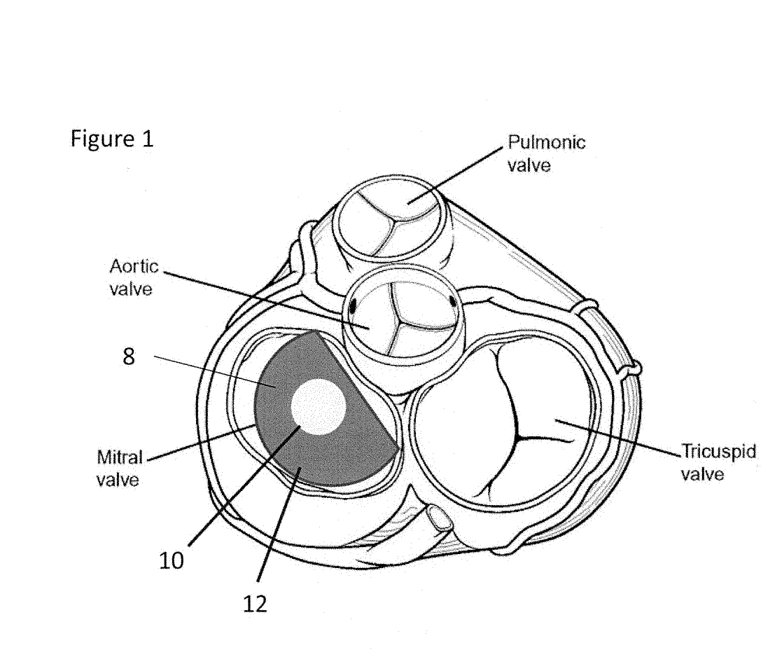

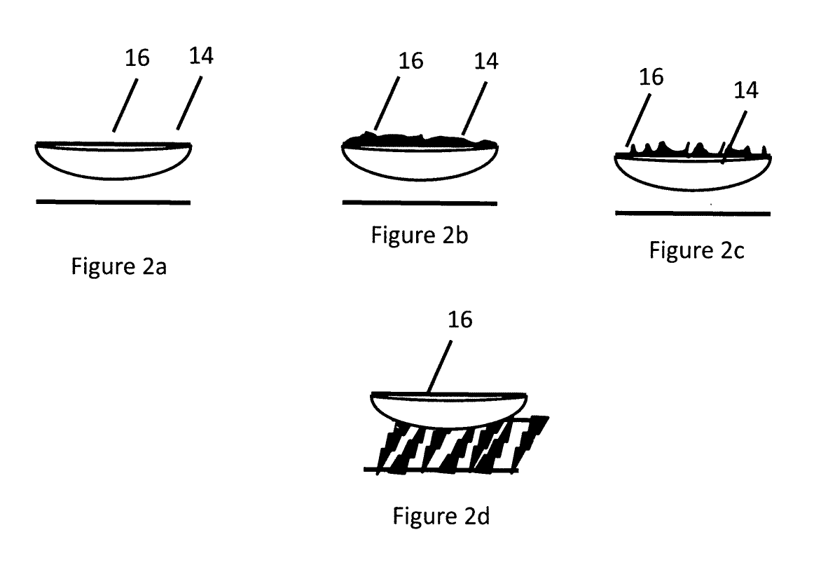

Example embodiments will now be described more fully with reference to the accompanying drawings. A cardiac prosthesis stabilization system according to the present teachings is shown in FIGS. 1-5b. Shown is a prosthetic support device 8 having an inner support ring 10 defining an internal valve support accepting surface 12. As shown in FIGS. 2a-2d, an outer support ring 14 having an outer surface 16 that is configured to fixably engage with cardiac muscle 18. An elastic member 20 is disposed between the outer support ring 14 and the inner support ring 10. The elastic member 20 provides a fluidly (blood) impermeable surface 22.

The outer support ring 14 which can be circular or D-shaped, is elastically deformable during cardiac systole. The elastic member 20 which can be a polymer, a fabric, or nitinol member and can have a fabric disposed about the plurality of metallic spring members is elastically deformable to minimize the deformation of the inner support ring 10 during the outer support ring 14 deformation during systole.

The outer support ring 14 outer surface can have a plurality of cardiac tissue engaging members. These cardiac tissue engaging members can be selected from micro spikes, calcium, a roughened surface, a metallic glass, and combinations thereof. The cardiac tissue engaging members are especially configured to engage the calcium like plaque materials which is typically found adjacent to valve surfaces.

As described above, the outer ring 14 can be a metallic self-expanding metallic titanium or nitinol ring having a circular or D shape. As shown in FIGS. 2a and 2b, the inner ring size in independent of the outer ring. Additionally, the outer ring 14 can be a PTFE toroid supporting ring that can be self-expanding or balloon expanding with the injection of liquid polymers. The size of the outer support ring whether metal or PTFE will be chosen based on the size of the valve or the LAA treated. The inflatable annulus/ring 14 made of PTFE membrane or other materials or of pericardium. The inflation liquid used to inflate the outer ring: liquid can undergo either a thermal transition or ultrasound curing into solid form once exposed to body, temperature. This liquid can be ether polymer bases or non-polymer based.

The toroidal outer ring can have an infusing port to inject the filling liquid into the inner space of the ring/this inflation access will have a detachment mechanism after completion of inflation and when stability of device is assured, although port will allow infusion of the toroid and also allow suctioning of liquid if needed.

The toroidal elastic member can be a PTFE membrane that preferably has several characteristics. A: the PTFE membrane is very loose and redundant. B. the outer surface of the PTFE membrane which will be in contact with cardiac tissue has rough surface that is engineered to provide the outer membrane with roughness that resembles the roughness and friction noted on a calcified valves, this roughness for example can be provided by mounting small or micro moldings on the outer surface of the annulus/ring that will be in contact with heart tissue when released in the body, C: in addition to these rough surface there are micro spikes that are also mounted on the outer surface of the annulus which is in contact with heart.

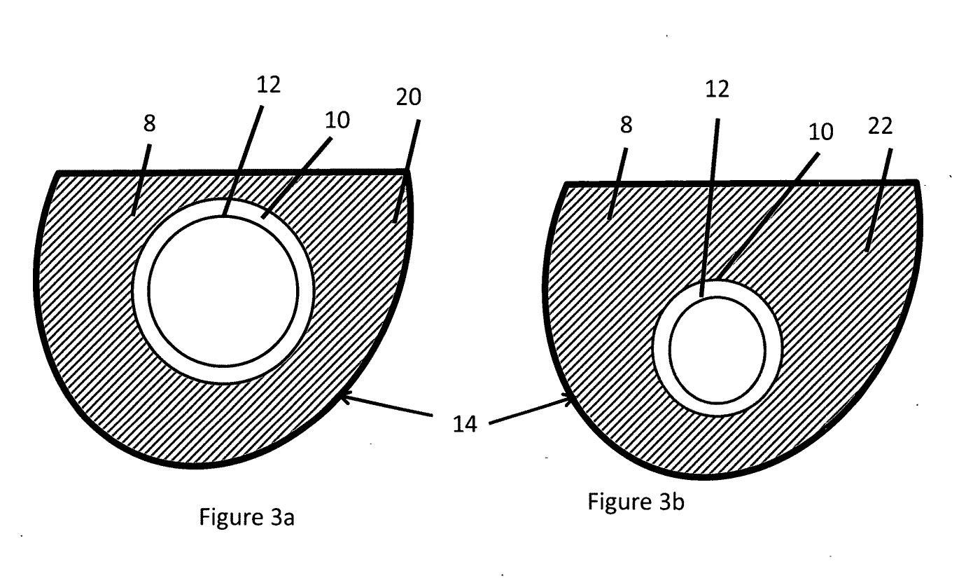

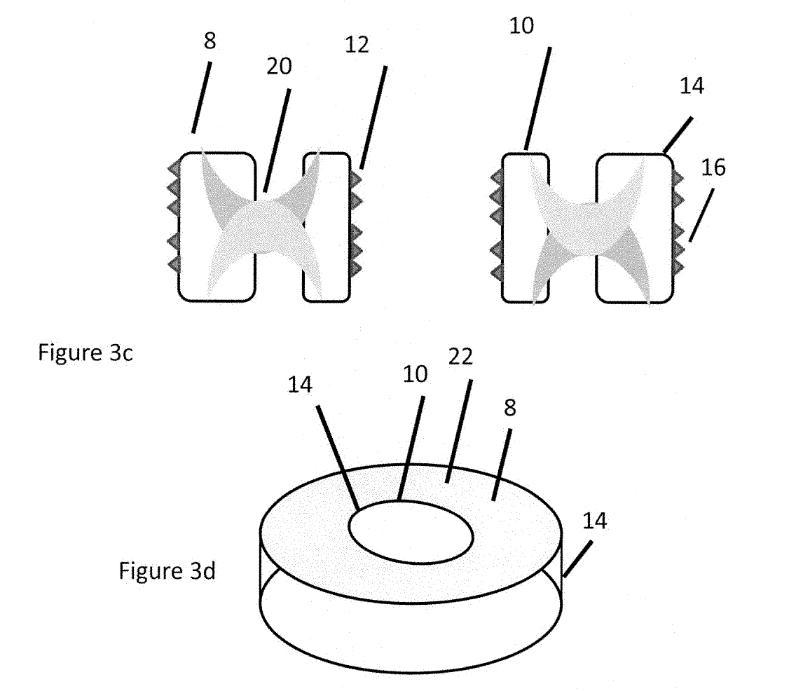

As shown in FIGS. 3a-3d, fabric material can be disposed about the outer and inner support rings as well as the elastic members 20. Optionally, the elastic member 20 can have a thickness which is smaller than the thickness of the outer and inner support rings.

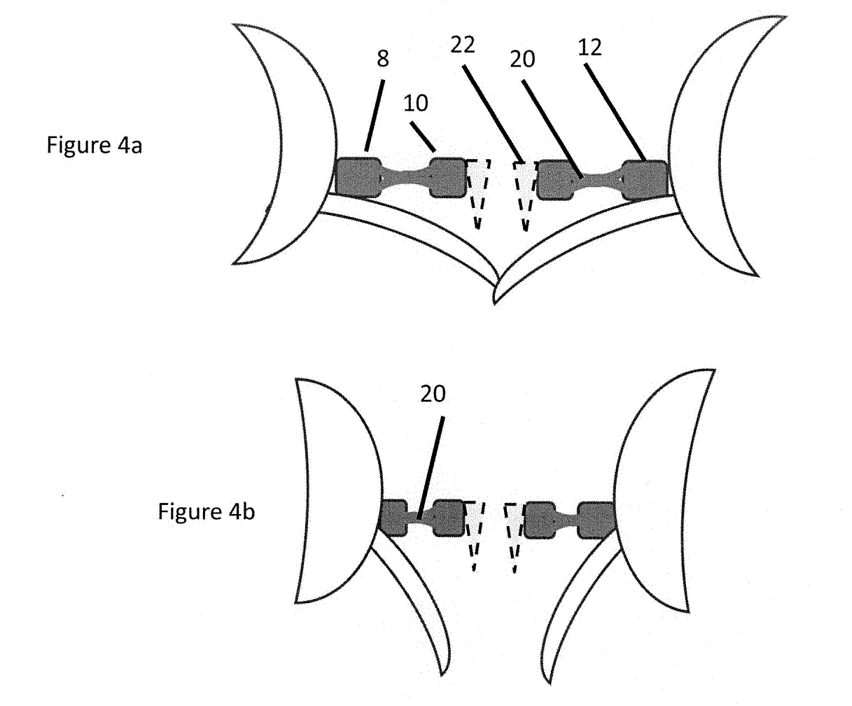

As shown in FIGS. 4a and 4b, the cardiac prosthesis stabilization system can include a stent supported valve 22 coupled to the inner support ring. This valve 22 can be configured to augment the natural function of a mitral or other cardiac valve, or can be used to entirely remove mitral valve tissue. As shown, the inner support ring defines an internal valve support accepting surface. The outer support ring having an outer surface configured to fixably engage with cardiac muscle. The elastic member is disposed between the outer support ring and the inner support ring, the elastic member providing a fluidly impermeable surface. The outer support ring is elastically deformable during cardiac systole and the elastic member is elastically deformable to minimize the deformation of the inner support ring during systole. As can be seen in FIG. 4b, when the outer ring is compressed, the inner ring remains substantially undeformed.

The device disclosed above allows stability, anchoring and sealing of devices used to treat valvular regurgitation in a noncalcified or calcified valvular disease or to seal and close left atrial appendage.

The valve material supported by the inner support ring 12 can be a circular or D shape sheath of pericardium or other artificial materials, this sheath of pericardium is sutured to the inner perimeter. The center of the elastic member is cut in one central circle or a shape to cover the coaptation area or several smaller circles, these circles will eventually allow unidirectional flow in the natural direction. The valve is connected to item 6 via cords made of pericardium or other artificial materials. In this regard, two sets of thin strings or cords in a + or triangle design or other designs. First set will be sutured inside the central cut circle of the elastic member.

The device 8 is inserted into the heart in a crimped form when released in its intended location either in a self-expanding method or balloon expanding mechanism it will assume its intended shape and will expand with it. Upon expansion, the outer ring 14 micro engaging structures and micro spikes as described above will be will expand to fill the spaces between the micro engaging structures and provide a homogeneous surface on the exterior.

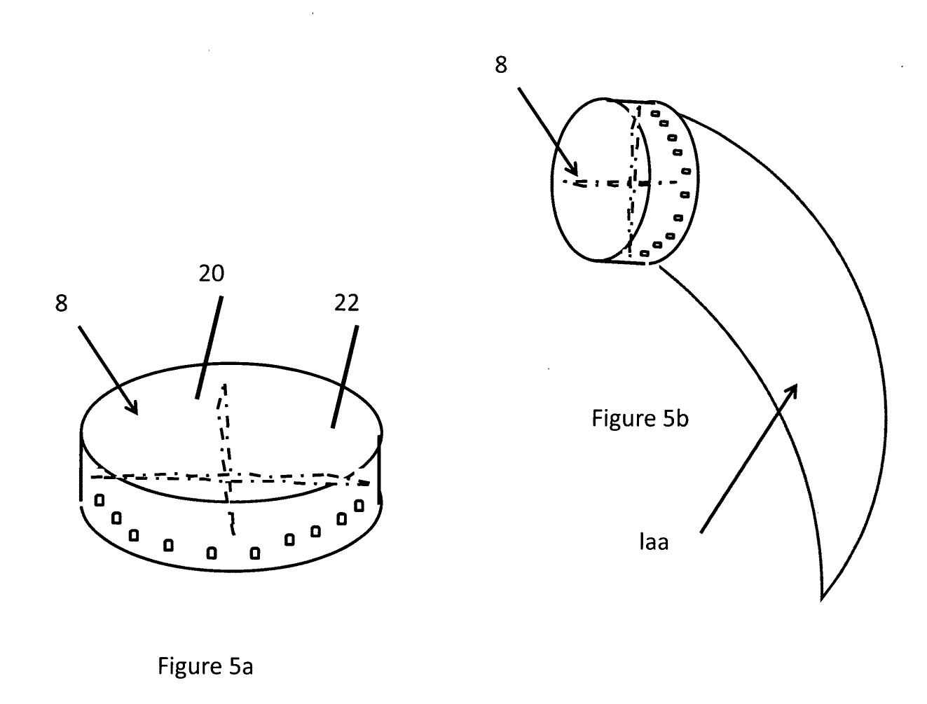

As shown in FIGS. 5a and 5b, the device 10 can be used as an LAA sealing and anchoring device. Unlike the device shown in FIG. 1, the central member is sealed. The device 8 can be inserted and coupled to the cardiac material in a manner similar to that disclosed above.

When faced with a mitral valve regurgitation, a physician can use the afore described system as is follows. First, the mitral valve annulus is sized. An appropriate exterior ring size some over-sizing chosen. The device can have sutured in it either a balloon expanding or self-expanding stent valve as a first option or can have the alternate valve structure as a second option. The device 8 is positioned next to the natural valve at the annular level on the atrial side. Once the physician is satisfied with the positioning, the outer ring is expanded to engage the cardiac tissue. For example, if a balloon expanding stent is used to expand the outer ring, or if the liquid is used to inflate toroid. If a self-expanding stent, inflating the outer ring will take place after expansion.

If the outer ring is a PTFE expandable toroid ring the presence of the roughness and the calcium like micro moldings and the micro spikes on the outer surface of the ring and under inflation pressure of the liquid 4 mechanical changes will happen. Preferable, the roughness of the PTFE membrane which is similar to a naturally calcified surface will provide strong stable friction contact with cardiac tissue. The micro spikes and under inflation pressure will embed into cardiac tissue providing more adherence of device with cardiac tissue. The loose, redundant PTFE membrane and as it fills with liquid it will occupy filled and fill the gaps spaces between the rough micro molds on the PTFE membrane preventing. These gaps if present can result in para-device leaks. The infused liquid will undergo thermal transition under impact of body temperature and transform into a solid form providing more stability/anchoring and sealing if the D device is implanted the inner component will move down during diastole allowing diastolic filling and will move up during systole stopping flow into LA.

To install an LAA device the LAA ostium is sized. An outer ring, corresponding size, some over sizing is needed. The device is deploy the device at the ostium or slightly distal to ostium of LAA in the usual fashion keeping in mind the importance of coaxially. Once satisfied with positioning the outer ring is expanded. The mechanical 4 changes will take place as described in the valve device leading to stability of the device with strong anchoring and sealing. The device 10 can sutured to the inner surface of the the LAA.

The Cardiac Prosthesis Stabilization System can be inserted by a physician using the following steps: 1—Device for the mitral valve indication can be introduced either trans-apically or after trans-septal puncture; 2—Device for aortic valve insufficiency can be used via retrograde femoral artery approach; 3—Device for tricuspid valve regurgitation can be applied via femoral vein approach or right internal jugular vein approach; and 4—Device for LAA closure can be performed after a trans-septal puncture approach. Additionally: can be described to treat venous insufficiency.

Mimic calcified aortic stenosis pathology (manufactured calcium like deposits with similar physical characteristics to calcium will provide adequate friction and capture between the ring and the native annulus tissue and between the ring and the stent frame). Oversized anchoring nitinol ring. PTFE ring (tube) surrounding the Nitinol wire which can be filled with polymer that will undergo thermal change to a solid form upon exposure to body temperature. Micro spikes (can under pressure embed in native tissue. Oversized stent in relation to ring.

By oversizing outer ring either with elastic size or by injecting liquid polymer and before undergoing thermal hardening will push PTFE membrane outward to seal all periphery device empty spaces and preventing PVL. After undergoing thermal transformation the presence of pressurized hardened polymer materials will add more anchoring force between the ring and native annulus. The presence of a layer of polymer between the dynamic annulus and the stent frame will protect the stent frame from the direct deforming force of the dynamic annular morphological changes.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A cardiac prosthesis stabilization system comprising:
an inner support ring defining an internal valve support accepting surface; an outer support ring having an outer surface configured to fixably engage with cardiac muscle;
an elastic member disposed between the outer support ring and the inner support ring, the elastic member providing a fluidly impermeable surface, wherein the outer support ring is elastically deformable during cardiac systole and the elastic member is elastically deformable to minimize the deformation of the inner support ring during systole.

2. The cardiac prosthesis stabilization system according to claim 1 wherein the elastic member is a polymer.

3. The cardiac prosthesis stabilization system according to claim 1 wherein the elastic member comprises a plurality of metallic spring members.

4. The cardiac prosthesis stabilization system according to claim 3 wherein the elastic member comprises one of a polymer member and a fabric disposed about the plurality of metallic spring members.

5. The cardiac prosthesis stabilization system according to claim 1 wherein the outer support ring comprises a plurality of cardiac tissue engaging members.

6. The cardiac prosthesis stabilization system according to claim 5 wherein the outer support ring comprises a plurality of cardiac tissue engaging members selected from micro spikes, calcium, a roughened surface, a metallic glass, and combinations thereof.

7. The cardiac prosthesis stabilization system according to claim 5 comprising a fabric tube disposed about a portion of the outer support ring.

8. The cardiac prosthesis stabilization system according to claim 7 wherein the fabric tube is disposed about a portion of the inner support ring.

9. The cardiac prosthesis stabilization system according to claim 1 further comprising a stent supported valve coupled to the inner support ring.

10. A cardiac prosthesis stabilization system comprising:
an inner support ring defining an internal valve support accepting surface; an outer support ring having an outer surface configured to fixably engage with cardiac muscle, the outer surface having a plurality of cardiac engagable surfaces;
a plurality of elastic members disposed between the outer support ring and the inner support ring, the elastic members providing a blood impermeable surface, wherein the outer support ring is elastically deformable during cardiac systole and the elastic member is elastically deformable to minimize the deformation of the inner support ring during systole.

11. The cardiac prosthesis stabilization system according to claim 10 wherein the outer support ring is D-shaped.

12. The cardiac prosthesis stabilization system according to claim 10 wherein the elastic members comprises a plurality of metallic spring members.

13. The cardiac prosthesis stabilization system according to claim 10 wherein the elastic members comprise one of a polymer membrane and a fabric disposed about the plurality of metallic spring members.

14. The cardiac prosthesis stabilization system according to claim 10 wherein the plurality of cardiac tissue engaging surfaces selected from micro spikes, calcium, a roughened surface, a metallic glass, and combinations thereof.

15. The cardiac prosthesis stabilization system according to claim 10 comprising a fabric member disposed about a portion of the outer and inner support rings.

16. The cardiac prosthesis stabilization system according to claim 10 wherein the inner support ring is elastically deformable.

17. The cardiac prosthesis stabilization system according to claim 10 further comprising a valve coupled to the inner support ring.

18. The cardiac prosthesis stabilization system according to claim 10 wherein at least one of the outer support ring, inner support ring and elastic members are formed of nitinol.
